(19) United States
(12) Patent Application Publication
(10) Pub. No.: US 2025/0257017 A1
(43) Pub. Date: Aug. 14, 2025

(54) METHOD FOR CONTROLLING A PROCESS COMPRISING A STEAM SYSTEM COUPLED TO A REACTOR SYSTEM

(71) Applicants: JOHNSON MATTHEY DAVY TECHNOLOGIES LIMITED, London (GB); BP PLC, London, Greater London (GB)

(72) Inventors: Robert Miles BAKER, London (GB); Colin TAMSETT, London (GB); Matthew Barry TURNBULL, Hull (GB)

(21) Appl. No.: 18/992,009

(22) PCT Filed: Aug. 2, 2023
(86) PCT No.: PCT/GB2023/052044

§ 371 Date: Jan. 7, 2025

(30) Foreign Application Priority Data
Sep. 9, 2022 (GB) 2213201.3
May 9, 2023 (GB) 2306773.9

(51) Int. Cl. C07C 1/04; B01J 8/00; B01J 8/06

(52) U.S. Cl. CPC C07C 1/042; B01J 8/001; B01J 8/067; B01J 2208/00168; B01J 2208/0061; B01J 2208/00637; B01J 2208/065

(57) ABSTRACT

A method for controlling a process comprising a steam system coupled to a reactor system, wherein the steam system comprises a steam vessel that feeds a stream of liquid water under pressure to the reactor system to cool the reactor system, thereby generating a steam stream, and receives the steam stream from the reactor system, the method comprising the steps of (i) obtaining a first total liquid level measurement in the steam vessel using an inferred level device, (ii) obtaining a second total liquid level measurement in the steam vessel using a direct level measurement device, (iii) calculating a difference between the first and second total liquid level measurements using a control system, and (iv) initiating an alarm using the control system when the difference between the first and second total liquid level measurements is ≥1% of the lower of the first and second total liquid level measurements.

This invention relates to a method for controlling a process comprising a steam system used to provide cooling to a reactor system, in particular for a reactor system used for the production of hydrocarbons in a Fischer-Tropsch process.

The Fischer-Tropsch process involves a series of catalysed chemical reactions in a reactor system that produce a variety of hydrocarbons, having the formula (CₙH₂ₙ₊₂) from a feed gas comprising hydrogen and carbon monoxide. The process may be operated in one or more Fischer-Tropsch reactors using iron-or cobalt-based catalysts at pressures in the range of 0.1 to 10 MPa and temperatures in the range of 170 to 350° C. The process may be operated to generate waxy hydrocarbons, which may be further processed in downstream treatments into fuels.

The Fischer-Tropsch reactions are exothermic and various arrangements have been developed to prevent over-heating and damage to the Fischer-Tropsch reactor and catalyst. In some arrangements, a reactor system comprising a fixed bed of Fischer-Tropsch catalyst is cooled in heat exchange with boiling water under pressure. In some arrangements, the water may flow though coolant tubes within a bed of particulate catalyst. In other arrangements, catalyst tubes containing particulate catalyst may be bathed by the water. In all cases, heat is transferred to the water, which boils generating steam. A steam system is therefore typically coupled to the reactor system to provide the water and receive the steam generated by the exchange of heat within the reactor system.

There is a risk that during a period of operation where the steam system is operating at lower pressure than the reactor system, hydrocarbon product of the Fischer-Tropsch reactions may leak into the steam system. The steam system will typically comprise a steam vessel, often described as a steam drum, that feeds the water under pressure to the reactor system and receives the steam from the reactor system. Accordingly, the steam vessel contains both steam and liquid water.

The Applicants have realised that due to the lower density of hydrocarbons (than water), these can accumulate in the steam vessel. A long-term accumulation of hydrocarbons in the steam vessel may undermine the efficacy of conventional steam-drum level monitoring instruments, as these will usually operate by measuring displacement or pressure differential between two heights within the drum. If a difference between the measured liquid level and water level occurs, there is a risk of insufficient cooling of the reactor, leading to potential run-away and catalyst or reactor damage.

The Applicants have found that using a combination of distinct techniques can improve the operation and control of the process.

Accordingly, the invention provides a method for controlling a process comprising a steam system coupled to a reactor system, wherein the steam system comprises a steam vessel that feeds a stream of liquid water under pressure to the reactor system to cool the reactor system, thereby generating a steam stream, and receives the steam stream from the reactor system, said method comprising the steps of (i) obtaining a first total liquid level measurement in the steam vessel using an inferred level device, (ii) obtaining a second total liquid level measurement in the steam vessel using a direct level measurement device, (iii) calculating a difference between the first and second total liquid level measurements using a control system, and (iv) initiating an alarm using the control system when the difference between the first and second total liquid level measurements is ≥1% of the lower of the first and second total liquid level measurements.

The invention includes a method for controlling a process comprising a steam system coupled to a reactor system, wherein the steam system comprises a steam vessel that feeds a stream of liquid water under pressure to the reactor system to cool the reactor system, thereby generating a steam stream, and receives the steam stream from the reactor system, said method comprising the steps of (i) obtaining a first total liquid level measurement in the steam vessel using an inferred level device, (ii) obtaining a second total liquid level measurement in the steam vessel using a direct level measurement device, (iii) calculating a difference between the first and second total liquid level measurements using a control system, and (iv) initiating an alarm using the control system when the difference between the first and second total liquid level measurements is ≥5% of the lower of the first and second total liquid level measurements.

The invention may be applied where the reactor system is operated at a higher pressure than the steam system, but it is also possible to operate the invention where the pressures of the systems are the same or even where the pressure of the steam system is greater than the pressure of the reactor system.

The temperature of the liquid water coolant provided by the steam system may be in the range 150 to 250° C. The pressure of the liquid coolant and steam in the steam vessel may be in the range 0.4 to 4.0 MPa (a).

The reactor system may be a Fischer-Tropsch reactor system including a Fischer-Tropsch catalyst, but the invention may be applied to any exothermic reactor system coupled to a steam system that provides cooling, if so desired.

The steam vessel contains both steam and liquid water. The method uses two different types of measurement device. This overcomes the deficiencies of using two devices of the same type.

The method includes obtaining a first total liquid level measurement in the steam vessel using an inferred level device. The inferred level device uses differences in densities or pressures within the vessel to measure by inference the total level of the liquid in the steam vessel. Such devices include positive displacement or differential pressure devices. Such devices are used in the chemical industry and are commercially available. The level of the liquid is the total liquid level, because the liquid may comprise both liquid water and another liquid, which may be immiscible with water. In a preferred embodiment, the total liquid level comprises a total of a liquid water level and an immiscible liquid level, such as a liquid hydrocarbon level. Where the immiscible liquid is a hydrocarbon or mixture of hydrocarbons, such as a Fischer Tropsch liquid, this will typically have a lower density than water and so form a layer on top of the liquid water in the steam vessel. The level detected therefore will be the level of the hydrocarbon on top of the liquid water in the steam vessel. The presence of a liquid hydrocarbon layer can create an error in an inferred level device measurement because the level inferred is typically based on the density of water. The presence of a lower density liquid therefore causes an inferred level device to read a level lower than the true level, because the weight of liquid that it measures will be less.

The method also includes simultaneously or sequentially obtaining a second total liquid level measurement in the steam vessel using a direct level measurement device. The direct level measurement device may use direct scanning methods to measure the total the level of the liquid in the steam vessel. Such devices include an ultrasonic or a guided wave radar (GWR) device. Such devices are used in the chemical industry and are commercially available. Alternatively, a float level device may be connected to the steam vessel and used to directly measure the liquid water level using a magnet connected to the float to indicate the level.

Where the reactor system comprises a Fischer-Tropsch reactor, using both an inferred level device and a direct level measurement device provides a particular benefit, which is that the level readings will tend to diverge if hydrocarbons start to build up in the liquid being measured.

In some arrangements, a guided-wave radar (GWR) instrument may be provided on the steam vessel in addition to an existing displacement or differential pressure instrument.

The method includes a step of comparing the first and second total liquid level measurements using a control system to calculate a difference between the first and second total liquid level measurements. The comparison comprises subtracting the smaller or lower liquid level measurement from the larger or higher liquid measurement. In this way, the difference will be a positive number. The comparison may make use of statistical techniques known in the art, such as n-point moving averages over time of the two values being compared, where n is a number between 2 and 100.

The method steps (i), (ii) and (iii) may be operated continuously or with regular frequent measurements every few seconds or minutes or hours. The calculation is performed using a control system. The control system may be any suitable control system used for controlling chemical reactors and processes. The control system may be a distributed control system (DCS). Distributed control systems are used to control many chemical processes and are available commercially.

In the method of the present invention includes calculating a difference between the first and second total liquid level measurements using the control system and initiating an alarm using the control system when the difference between the first and second total liquid level measurements is ≥1% or ≥5% of the lower of the first and second total liquid level measurements. If the difference is below this value, the control system does not initiate an alarm but rather simply repeats the previous steps to monitor the steam vessel. This may be performed continuously or periodically.

If the difference between the measurements is ≥1% or ≥5% of the lower of the first and second total liquid level measurements, an alarm is initiated so that an operator of the process can investigate the source of the difference. This may include, in response to the alarm, one or more steps of: monitoring the liquid water flow and temperature from the steam vessel; monitoring the temperature of a reaction vessel in the reactor system; and; monitoring the chemical composition of the liquid in the steam vessel, for example by taking and analysing a liquid sample for pH.

A display system having a visible or audible alarm may be connected to the control system. Connection of the control system to any alarm system may be wireless or by a direct hard-wired connection.

The operator may perform a shutdown of the process using the reactor system. A shut down of the reactor system may become necessary when the calculated difference is ≥1% or 5% or higher, especially 5% or higher. Therefore, in some embodiments the method includes a further step of shutting down the reactor system to prevent overheating of the reactor and catalyst. Shut-down methods for reactor systems are known. Shut down methods for Fischer-Tropsch reactor systems are described for example in GB2223237A, U.S. Pat. No. 10,329,492, and WO2022/11784A1, the contents of which are incorporated herein by reference. In such methods the steam vessel may be depressurised to cool the liquid water coolant and the supply of fresh synthesis gas stopped. Alternatively, or in addition, an inert gas, such as nitrogen gas, may be injected into the reactor system if desired.

The method may usefully be applied to process including a Fischer-Tropsch reactor containing a cooled Fischer-Tropsch catalyst fed with a reactant gas mixture and operated in a loop.

The reactant gas mixture fed to the Fischer-Tropsch reactor typically comprises a synthesis gas, plus a recycle gas, which is recovered from the Fischer-Tropsch reactor product stream. The synthesis gas for a Fischer-Tropsch process comprises hydrogen and carbon monoxide. The recycle gas will typically contain unreacted synthesis gas, carbon dioxide and potentially light hydrocarbons.

The Fischer-Tropsch process involves a series of chemical reactions that produce a variety of hydrocarbons, ideally having the formula (CₙH₂ₙ₊₂). The more useful reactions produce alkanes from the reactant gas mixture as follows:

(2n+1)H₂+nCO→CₙH₂ₙ₊₂+nH₂O,

where n is typically 5-100 or higher, with preferred products having n in the range 10-20.

The Fischer-Tropsch reactor is typically operated in a synthesis loop, i.e. the reactant gas mixture is fed to the Fischer-Tropsch reactor where it reacts over the Fischer-Tropsch catalyst to form a product mixture comprising liquid and gaseous hydrocarbons, steam and unreacted gases. The product gas mixture is cooled after leaving the Fischer-Tropsch reactor to condense steam and facilitate recovery of the liquid hydrocarbons. A portion of the unreacted gas, optionally after separation of light hydrocarbons, is returned to the Fischer-Tropsch reactor as the recycle gas thereby forming a synthesis loop. The recycle gas is combined with the synthesis gas to form the reactant gas mixture outside of the Fischer-Tropsch reactor, which allows for more efficient temperature control of the feed to Fischer-Tropsch reactor. Operating the Fischer-Tropsch reactor in a loop enhances the conversion efficiency of the process. To prevent a build-up of inert gases, a purge may be taken from the loop as a Fischer-Tropsch tail gas, which may be subjected to further processing.

The Fischer-Tropsch reactor may be operated at pressures in the range 10 to 100 bar abs (0.1 to 10MPa) and temperatures in the range 170 to 350° C. Operation over cobalt catalysts may be at 20-50 bar abs and 200-320° C. The gas-hourly-space velocity (GHSV) for continuous operation may be in the range 1000 to 25000 hr⁻¹.

The Fischer-Tropsch reactor contains a Fischer-Tropsch catalyst cooled indirectly by water under pressure. The Fischer-Tropsch catalyst may be provided as a bed though which coolant-bearing tubes or plates are placed, or the catalyst may be provided in a plurality of reactor tubes that are bathed in coolant flowing around their outsides. The latter reactor technology is preferred.

Any Fischer-Tropsch catalyst may be used, but iron and cobalt Fischer-Tropsch catalysts are preferred. Cobalt-based Fischer-Tropsch catalysts are preferred over iron-based catalysts due to their lower carbon dioxide selectivity. In a particularly preferred arrangement, the Fischer-Tropsch catalyst is used in combination with a catalyst carrier suitable for use in a tubular Fischer-Tropsch reactor where the catalyst carrier containing the catalyst is disposed within one or more tubes that are cooled by circulating water under pressure. By “catalyst carrier” we mean a catalyst container, for example in the form of a cup or can, configured to allow a gas and/or liquid to flow into and out of the carrier and through a bed of the catalyst or catalyst precursor disposed within the carrier. Any suitable catalyst carrier may be used. In one arrangement, the catalyst carrier is that described in WO2011/048361, the contents of which are incorporated herein by reference. In an alternative arrangement, the catalyst carrier may include a catalyst monolith as disclosed in WO2012/136971, the contents of which are also incorporated herein by reference. In yet another alternative arrangement, the catalyst carrier may be that disclosed in WO2016/050520, the contents of which are also incorporated herein by reference. In preferred embodiments, the reactor system comprises a tubular Fischer-Tropsch reactor in which catalyst carriers containing a Fischer-Tropsch catalyst are disposed within one or more tubes cooled by a cooling medium.

The invention is further described by reference to the drawing in which:

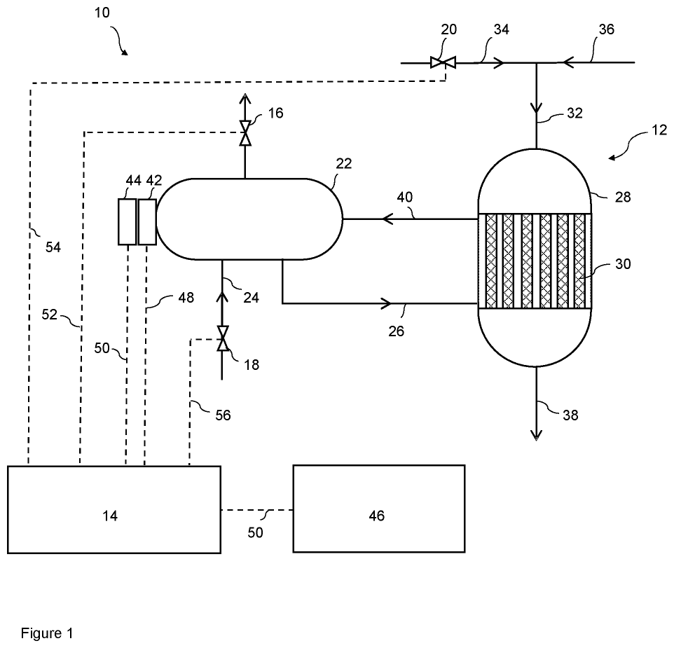

FIG. 1 is a depiction of one embodiment of a system to which the method of the present invention may be applied.

It will be understood by those skilled in the art that the drawings are diagrammatic and that further items of equipment such as feedstock drums, pumps, vacuum pumps, compressors, gas recycling compressors, temperature sensors, pressure sensors, pressure relief valves, control valves, flow controllers, level controllers, holding tanks, storage tanks and the like may be required in a commercial plant. Provision of such ancillary equipment forms no part of the present invention and is in accordance with conventional chemical engineering practice.

In FIG. 1 a steam system 10 is coupled to a Fischer-Tropsch reactor system 12. A DCS control system 14 controls the steam system and reactor system by means of valves 16, 18, 20. The steam system 10 comprises a steam vessel 22 fed with a stream of boiler feed water via line 24, that in turn feeds a stream of liquid water under pressure via line 26 to a Fischer-Tropsch reactor 28 where it is used to cool a plurality of Fischer-Tropsch catalyst-containing tubes 30. A feed gas 32, comprising a fresh synthesis gas stream 34 and a recycle gas stream 36 is fed to the reactor 28, where it reacts over the catalyst in the tubes 30 to generate hydrocarbon liquid products, which are recovered from the reactor, along with unreacted gas and by-product water as a product stream 38 for further processing. The fresh synthesis gas stream 34 and a recycle gas stream 36 are compressed by compressors (not shown). The pressure of the feed gas mixture 32 is greater than the pressure of the water under pressure fed via line 26. The recycle gas stream 36 is recovered from the product stream 38 using one or more gas-liquid separators (not shown).

The formation of the hydrocarbon liquids generates heat that converts a portion of the liquid water provided by line 26 to steam inside the reactor 28. A mixture of steam and liquid water is recovered from the reactor 28 and fed via line 40 to the steam vessel 22. The steam system 10 further comprises an inferred level device 42 that obtains a first total liquid level measurement in the steam vessel 22, and a direct level measurement device 44 that obtains a second total liquid level measurement in the steam vessel 22. The levels detected by the devices 42, 44 are communicated (shown by dashed lines 48 and 50) to the controller 14 that compares the first and second total liquid level measurements and calculates a difference between them. The control system 14 is connected (as shown by a dashed line 52) to a display system 46 having a visible and audible alarm. An alarm is initiated in the display system 46 using the control system 14 when the difference between the first and second total liquid level measurements is ≥5% of the lower of the first and second total liquid level measurements.

The control system 14 is connected (as shown by dashed lines 54, 56 and 58) to the valves 16, 18, 20 and upon instruction from the control system or operator, for example, based upon the temperature of the catalyst within the tubes 30, the valves 16, 18 and 20 may be adjusted to bring about a controlled shut down of the reactor system 12. For example, the valve 16 may be opened to depressurise the steam vessel 22 thereby reducing the temperature of the liquid water and quenching the Fischer-Tropsch reactions. Alternatively, or additionally, the feed of fresh synthesis gas may be stopped by closing valve 20. The circulating compressor continues to feed the recycle gas stream 32 to the reactor. Optionally, a pressure vessel (not shown) containing high-pressure nitrogen at a pressure greater than the feed gas 32 may be connected to the recycle gas line 36 or feed line 32 for use in case of emergency to inject nitrogen gas into the catalyst-filled tubes 30. The valve 18 controlling the feed of boiler feed water to the steam vessel 22 may also subsequently be closed to shut off the feed water.

1. A method for controlling a process comprising a steam system coupled to a reactor system, wherein the steam system comprises a steam vessel that feeds a stream of liquid water under pressure to the reactor system to cool the reactor system, thereby generating a steam stream, and receives the steam stream from the reactor system, said method comprising the steps of (i) obtaining a first total liquid level measurement in the steam vessel using an inferred level device, (ii) obtaining a second total liquid level measurement in the steam vessel using a direct level measurement device, (iii) calculating a difference between the first and second total liquid level measurements using a control system, and (iv) initiating an alarm using the control system when the difference between the first and second total liquid level measurements is ≥1% of the lower of the first and second total liquid level measurements, wherein the reactor system is a Fischer-Tropsch reactor system including a Fischer-Tropsch catalyst cooled indirectly by the stream of liquid water from the steam vessel, wherein the Fischer-Tropsch reactor system is operated at a higher pressure than the steam system, and wherein the difference between the first and second total liquid level measurements initiating the alarm is caused by hydrocarbon product of the Fischer-Tropsch reactor system leaking into the steam system and accumulating in the steam vessel.

2. (canceled)

3. The method according to claim 1, wherein the inferred level device is a positive-displacement device or a differential-pressure device.

4. The method according to claim 1, wherein the direct level measurement device is a guided-wave-radar device or a float device.

5. The method according to claim 1, wherein the method steps (i), (ii) and (iii) are operated continuously.

6. The method according to claim 1, wherein the method steps (i), (ii) and (iii) are performed by measurements every few seconds, minutes or hours.

7. The method according to claim 1, wherein the difference between the first and second total liquid level measurements is calculated using a time-averaged or statistical method.

8. The method according to claim 1, wherein the control system is a distributed control system.

9. The method according to claim 1, wherein a display system having a visible or audible alarm is connected to the control system.

10. The method according to claim 1, wherein in response to the alarm, the method further comprises one or more steps of: monitoring the liquid water flow and temperature from the steam vessel; monitoring the temperature of a reaction vessel in the reactor system; and; monitoring the chemical composition of the liquid in the steam vessel.

11. The method according to claim 1, wherein in response to the alarm, the method includes a further step of shutting down the reactor system.

12. (canceled)

13. The method according to claim 12, wherein the Fischer-Tropsch catalyst is provided as a bed though which water-bearing tubes or plates are placed, or the Fischer-Tropsch catalyst is provided in a plurality of reactor tubes that are water-cooled.

14. The method according to claim 1, wherein the Fischer-Tropsch catalyst is used in combination with a catalyst carrier in a tubular Fischer-Tropsch reactor where the catalyst carrier containing the Fischer-Tropsch catalyst is disposed within one or more tubes that are cooled by circulating water under pressure.

15. The method according to claim 1, wherein the alarm is initiated using the control system when the difference between the first and second total liquid level measurements is ≥5% of the lower of the first and second total liquid level measurements.
